(19) United States
(12) Patent Application Publication
(10) Pub. No.: US 2009/0209587 A1
(43) Pub. Date: Aug. 20, 2009

(54) REPAGLINIDE FORMULATIONS

(76) Inventors: Parag Prakash Borde, Jalna (IN); Praveen Kumar Bembaloor Subbappa, Coorg (IN); Ravinder Kodipyaka, Kaghaznagar (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/352,784

(22) Filed: Jan. 13, 2009

Related U.S. Application Data
(60) Provisional application No. 61/035,533, filed on Mar. 11, 2008.

(30) Foreign Application Priority Data
Jan. 14, 2008 (IN) 123/CHE/2008

(51) Int. Cl. A61K 31/451

(52) U.S. Cl. 514/331

(57) ABSTRACT

Processes for preparing solid pharmaceutical compositions comprising repaglinide or a derivative thereof, including a step of granulation. Embodiments include fluid bed granulation.

### INTRODUCTION

The present invention relates to solid pharmaceutical formulations containing repaglinide or pharmaceutically acceptable derivatives thereof.

Further, the present invention relates to processes of preparing solid pharmaceutical formulations containing repaglinide.

The present invention also relates to cost-effective processes for preparing stable pharmaceutical formulations.

Repaglinide has a chemical name S-(+)-2-Ethoxy-4-(2((3-methyl-1-(2-(1-piperidinyl)phenyl)butyl)amino)₂-oxoethyl) benzoic acid. It is used to treat diabetes. It belongs to the meglitinide class of insulin secretagogues, compounds that stimulate insulin release from the pancreas.

Repaglinide is a white to off-white powder with the molecular formula C₂₇H₃₆N₂O₄ and a molecular weight of 452.6. It is practically insoluble in water, and freely soluble in methanol and in methylene chloride. It has strong pH-dependent solubility and is highly lipophilic. Its structural formula is Formula I.

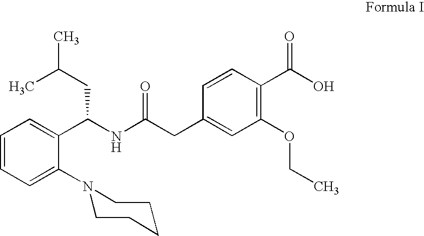

Repaglinide lowers the blood glucose levels by stimulating the release of insulin from the pancreas. This action is dependent upon functioning beta cells in the pancreatic islets. Insulin release is glucose-dependent and diminishes at low glucose concentrations.

Repaglinide closes ATP-dependent potassium channels in the beta-cell membrane by binding at characterized sites. The potassium channel blockade depolarizes the beta-cells, which leads to an opening of calcium channels. The increased calcium influx induces insulin secretion. The ion channel mechanism is highly tissue selective with low affinity for heart and skeletal muscle.

Repaglinide is currently marketed as PRANDIN® by Novo Nordisk. PRANDIN® tablets for oral administration are available in three strengths: 0.5 mg, 1 mg, and 2 mg of repaglinide.

PRANDIN® is indicated as an adjunct to diet and exercise to lower the blood glucose in patients with type I diabetes mellitus (non-insulin dependent diabetes mellitus, or NIDDM) and also as an adjunct in combination therapy with thiazolidinediones to lower the blood glucose in patients whose hyperglycemia cannot be controlled by diet and exercise.

Repaglinide is disclosed in U.S. Pat. Nos. 5,312,924 and RE37,035. U.S. Pat. No. 6,143,769 discloses the (S)-enanantiomer of repaglinide and its salts.

A need remains for a simple, cost-effective process for preparing replaglinide pharmaceutical formulations having commercially acceptable properties.

### SUMMARY

The present invention relates to solid pharmaceutical formulations comprising repaglinide or its derivatives.

Further the present invention relates to processes for preparing solid pharmaceutical formulations comprising repaglinide or its derivatives.

In one of the embodiments the invention includes stable solid pharmaceutical formulations comprising repaglinide or its derivatives.

In an embodiment the present invention includes formulation processes using granulation techniques such as granulation with a fluid bed granulator.

In an embodiment the present invention includes formulation processes using granulation techniques such as granulation with a rapid mixer granulator.

In an aspect the invention includes processes for preparing solid pharmaceutical formulations comprising repaglinide or a derivative thereof, in which an embodiment comprises dissolving repaglinide or a derivative thereof in a binder solution, and granulating other excipients with the binder solution.

In an aspect the invention includes processes for preparing solid pharmaceutical formulations comprising repaglinide or a derivative thereof, in which an embodiment comprises dry mixing repaglinide or a derivative thereof with at least one pharmaceutical excipient, and granulating the mixture with a binder solution.

In an embodiment the invention includes the use of top spray granulation in processes for preparing solid pharmaceutical formulations comprising repaglinide or its derivatives.

In an embodiment the invention includes the use of bottom spray granulation in processes for preparing solid pharmaceutical formulations comprising repaglinide or its derivatives.

In an embodiment the present invention includes formulations prepared using repaglinide having particle size distributions wherein D₉₀ is less than about 50 μm, less than about 20 μm, or less than about 7 μm.

In an embodiment the present invention includes formulations prepared using repaglinide having particle size distributions wherein D₅₀ is less than about 25 μm, less than about 15 μm, or less than about 4 μm.

In an embodiment the present invention includes formulations prepared using repaglinide having particle size distributions wherein D₁₀ is less than about 15 μm, less than about 5 μm, or less than about 2 μm.

In an embodiment the present invention relates to formulations prepared using particle size distributions of final blends comprising repaglinide and excipients, wherein about 0.1-5% of particles have sizes greater than about 250 μm, about 5-40% of particles have sizes greater than about 180 μm, and more than about 50% of particles have sizes greater than about 150 μm.

In another embodiment the invention includes pharmaceutical formulations comprising repaglinide or its derivatives wherein a relative standard deviation (RSD) in the content of repaglinide or its derivatives is not more than about 6.

In an embodiment the present invention relates formulations prepared using repaglinide having bulk densities in the range of about 0.1-0.3 g/ml.

In an embodiment the present invention relates formulations prepared using repaglinide having tapped densities in the range of about 0.2-0.4 g/ml.

In an embodiment the present invention relates to formulations prepared using repaglinide-containing final blends with excipients, having bulk densities in the range of about 0.5-0.8 g/ml.

In an embodiment the present invention relates to formulations prepared using repaglinide-containing final blends with excipients, having tapped densities in the range of about 0.6-0.9 g/ml.

In an embodiment the present invention relates to methods of using pharmaceutical formulations comprising repaglinide of the invention to treat non-insulin dependent diabetes mellitus.

An embodiment of the invention includes a process for preparing a pharmaceutical formulation, comprising granulating at least one pharmaceutically acceptable excipient with a solution or dispersion comprising repaglinide, or a derivative thereof, and optionally a basifying agent.

An embodiment of the invention includes a process for preparing a pharmaceutical formulation, comprising granulating at least one pharmaceutically acceptable excipient with a solution or dispersion comprising repaglinide, or a derivative thereof, and a basifying agent comprising meglumine, wherein granulation is conducted in a fluid bed granulator.

An embodiment of the invention includes a process for preparing a pharmaceutical formulation, comprising granulating at least one pharmaceutically acceptable excipient with a solution or dispersion comprising repaglinide, or a derivative thereof, and a basifying agent comprising meglumine, wherein granulation is conducted in a rapid mixer granulator.

### DETAILED DESCRIPTION

The present invention relates to solid pharmaceutical formulations comprising repaglinide or its derivatives.

Further the present invention relates to processes of preparing solid pharmaceutical formulations comprising repaglinide.

In an aspect, the invention relates to processes for preparing solid pharmaceutical formulations comprising repaglinide, an embodiment of a process comprising:

(i) Dry mixing repaglinide or a salt thereof with at least one pharmaceutical excipient.

(ii) Granulating the mixture obtained in step (i) with a binder solution by granulation either in a fluid bed granulator or in a rapid mixer granulator.

A pharmaceutically acceptable derivative includes any pharmaceutically acceptable salt, ester, or salt of an ester of repaglinide, and is not restricted to any particular polymorphic form of a compound.

In an embodiment the present invention includes pharmaceutical formulations comprising repaglinide or its pharmaceutically acceptable derivative in concentrations of about 1% to about 95%, or about 25% to about 90%, or from about 50% to about 85%, by weight of the total composition.

Repaglinide is observed to have pH-dependent solubility. Repaglinide has two ionizable groups and the solubility at any given pH is the sum of the concentrations of each different species in saturated solution. The solubility profile is U-shaped for zwitterionic compounds such as repaglinide.

Repaglinide, being an unstable molecule, undergoes many degradation reactions and the most common degradation is by hydrolysis in basic conditions. Impurity A and Impurity C are the major degradants formed by hydrolysis. Some degradation products of repaglinide include:

“Impurity A” having a chemical name 4-(carboxymethyl)-2-ethoxybenzoic acid and represented by Formula II.

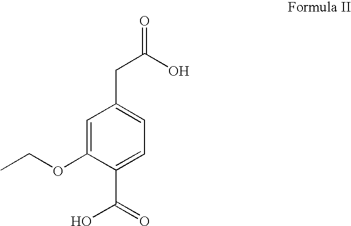

“Impurity B” having a chemical name 3-ethoxy-4-(ethoxycarbonyl) phenyl]acetic acid and represented by Formula III.

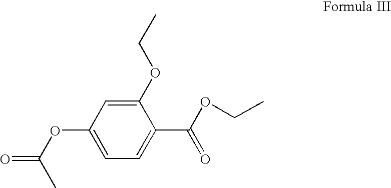

“Impurity C” having a chemical name 3-methyl-1-(2-piperidin-1-yl-phenyl)-butyl-1-amine and represented by Formula IV.

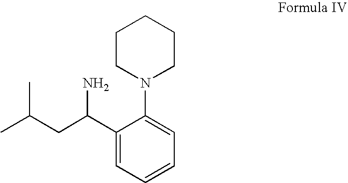

“Impurity D” having a chemical name ethyl 2-ethoxy-4-[2-[[(1S)-3-methyl-1-[2-(piperidin-1-yl)phenyl]butyl]amino]-2-oxoethyl]benzoate and represented by Formula V.

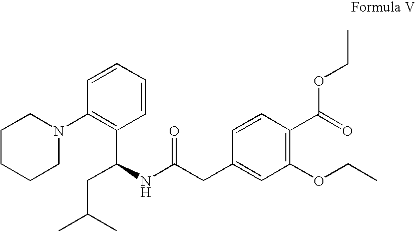

“Impurity E” having a chemical name 2-ethoxy-4-[2-[[(1R)-3-methyl-1-[2-(piperidin-1-yl) phenyl] butyl]] amino]-2-oxoethyl] benzoic acid and represented by Formula VI.

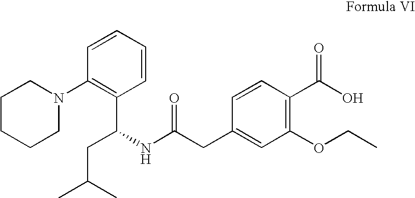

“Cyclic impurity I” having a chemical name 2-ethoxy-4-(2-((4aS, 6S)-2,3,4,4a-tetrahydro-6-isobutyl-1H-pyrido[1,2-a] quinazolin-5 (6H)-yl)-2-oxoethyl) benzoic acid and represented by Formula VIII.

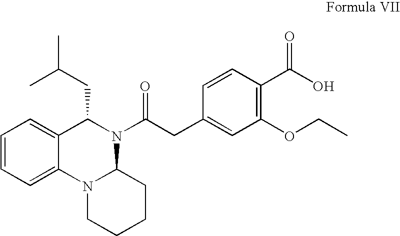

“Cyclic impurity II” having a chemical name 2-ethoxy-4-(2-((4aR, 6S)-2,3,4,4a-tetrahydro-6-isobutyl-1H-pyrido[1,2-a]quinazolin-5 (6H)-yl)-2-oxoethyl) benzoic acid and represented by Formula VIII.

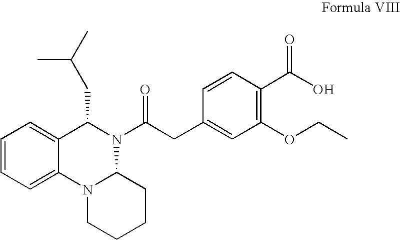

A “desethyl impurity” having a chemical name S (+) 2-hydroxy-4(2((3-methyl-1-(2-(1-piperidinyl) phenyl)-butyl) amino)-2-oxoethyl) benzoic acid and represented by Formula IX.

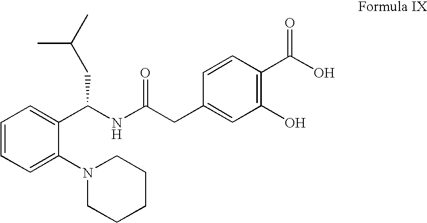

“Specified impurity III” having a chemical name 4-{[1-(2-Amino-phenyl)-3-methyl-butylcarbamoyl]-methyl}-2-ethoxy-benzoic acid and represented by Formula X.

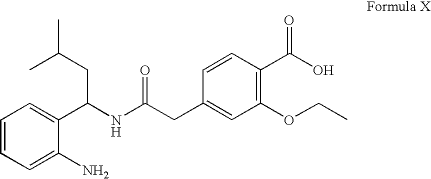

Two uncharacterized impurities called “specified impurity I” and “specified impurity II” have also been formed.

In an embodiment the present invention includes pharmaceutical formulations comprising repaglinide, wherein the formulations comprise less than about 2%, or less than about 1%, or less than about 0.5%, of cyclic impurity I. In an embodiment the present invention includes pharmaceutical compositions comprising repaglinide, wherein the compositions comprise less than about 2%, or less than about 1%, or less than about 0.5%, of cyclic impurity II.

In one of the embodiment the invention includes stable pharmaceutical formulations wherein total impurities are not more than about 4%, or not more than about 3%. All of the foregoing impurity contents are expressed as percentages of the label drug content in the pharmaceutical formulations.

In an embodiment, the present invention includes stable pharmaceutical formulations comprising repaglinide.

In yet another embodiment the invention includes stable pharmaceutical formulations wherein the water content is less than about 8%, or less than about 6% by weight. The water content of a formulation can conveniently be determined using techniques such as weight loss during drying and Karl Fischer moisture analysis.

In embodiments, processes to prepare repaglinide formulations of the present invention include wet granulation using a fluid bed granulator or a rapid mixer granulator.

Wet granulation comprises weighing solid components, preparation and addition of binder solution, screening, drying and blending with lubricants before tableting.

Various parameters impacting granulation processes are compressibility, flow properties, and particle sizes of the active ingredient and final blend with excipients, and other properties of the excipients such as moisture content (determined by Karl Fischer (KF) apparatus or infrared moisture balance), particle size (determined by sieve analyzer or devices such as a light scattering particle size analyzer, such as those from Malvern Instruments Limited, Malvern, Worcestershire, United Kingdom), bulk density and tapped density, flow properties (determined by a Flowdex apparatus), compressibility, etc.

When a potent drug such as repaglinide is present in a low concentration in the total formulation, it is necessary to ensure that the drug is uniformly distributed in the formulation so that there is acceptable variation in the dose that is administered in each unit dosage form. The uniformity of content of drug is expressed in terms of relative standard deviation.

A uniform distribution of the drug in the formulation may be achieved by many ways such as by using drug having uniform particle size distributions, by optimizing different steps of processing of the composition such as mixing and blending the active and inactive excipients, or by appropriate selection of excipients, etc.

In an embodiment the present invention provides pharmaceutical formulations comprising repaglinide, wherein said formulations have uniformity of content of repaglinide such that the relative standard deviation of repaglinide content is not more than about 6.

Since repaglinide is a water-insoluble compound, it is expected that particle size reduction would improve the solubiltiy. The particles of repaglinide used for manufacturing the present formulations are in a micronized form in order to improve the dissolution profile.

The percentages of particles with different sizes that exist in a total powder is the particle size distribution. It is represented in certain ways. Particle size is the maximum dimension of a particle, frequently expressed in μm. Particle size distributions can be expressed in terms of, D₁₀, D₅₀, D₉₀ and D_{[4,3]}. The D₁₀, D₅₀ and D₉₀ represent the 10th, median or the 50th percentile, and the 90th percentile of the particle size distribution, respectively, as measured by volume. That is, the D₁₀, D₅₀, D₉₀ are values of the distribution such that 10%, 50%, or 90% of the particles have a volume of this value or less, or is the percentage of particles smaller than that size. D₅₀ is also known as median diameter of particle. It is one of the important parameters representing characteristics of particles of powders. For a sample, if D₅₀=5 μm, it means that 50% of the particles are smaller than 5 μm. Similarly if D₁₀=5 μm, 10% of the particles are less than or equal to 5 μm, and if D₉₀=5 μm, 90% of the particles have sizes less than or equal to 5 μm. D_{[4,3]} represents the volume moment mean of the particles, or the volume weighted particle size.

Particles of repaglinide or its derivatives having defined size ranges are used in the pharmaceutical formulations of the present invention. These specified particle sizes have a beneficial influence on content uniformity and the release profile of the composition.

In an embodiment, the invention includes pharmaceutical formulations comprising repaglinide having defined particle size distributions.

In an embodiment the present invention includes formulations prepared using repaglinide having particle size distributions wherein D₉₀ is less than about 50 μm, less than about 20 μm, or less than about 7 μm.

In an embodiment the present invention includes formulations prepared using repaglinide having particle size distributions wherein D₅₀ is less than about 25 μm, less than about 15 μm, or less than about 4 μm.

In an embodiment the present invention includes formulations prepared using repaglinide having particle size distributions wherein D₁₀ is less than about 15 μm, less than about 5 μm, or less than about 2 μm.

This micronized particle size distribution for a potent drug such as repaglinide is useful to get content uniformity in the composition.

In an embodiment the present invention relates to pharmaceutical formulations prepared using particle size distributions of final powder blends comprising repaglinide or its derivatives and excipients, wherein about 0.1-5% of particles have sizes greater than about 250 μm, about 5-40% of particles have sizes greater than about 180 μm, and more than about 50% of particles have sizes greater than about 150 μm.

In another embodiment the invention includes pharmaceutical formulations prepared using repaglinide or its derivatives wherein percent relative standard deviation (RSD) of the concentrations of repaglinide or its derivatives is not more than about 6.

In an embodiment the present invention relates to formulations prepared using repaglinide having bulk densities in the range of about 0.1-0.3 g/ml.

In an embodiment the present invention relates to formulations prepared using repaglinide having tapped densities in the range of about 0.2-0.4 g/ml.

In an embodiment the present invention relates to formulations prepared using repaglinide-containing final blends with excipients, having bulk densities in the range of about 0.5-0.8 g/ml.

In an embodiment the present invention relates to formulations prepared using repaglinide-containing final blends with excipients, having tapped densities in the range of about 0.6-0.9 g/ml.

In embodiments of the present invention the pharmaceutical formulations may be solid dosage forms such as tablets, capsules, granules, etc.

Pharmaceutical formulations of the present invention further contain other pharmaceutically acceptable excipients, which include but are not limited to any one or more of diluents fillers, binders, glidants, lubricants, basifying agents, surfactants, colorants, flavors, and solvents.

### Diluents or Fillers:

Various useful fillers or diluents include but are not limited to starches, lactose, mannitol, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

### Binders:

Various useful binders include but are not limited to hydroxypropylcelluloses (Klucel™ LF), hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starches.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropyl celluloses. Examples of low-substituted hydroxypropyl celluloses include but are not limited to low-substituted hydroxypropyl cellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches. Resins may also be used as disintegrants. Nonlimiting examples of useful resins include Amberlite® IR-120 Plus (H), Amberlite® IR-120 Plus, Amberlite® IRP-69, Amberlite® 15, Amberlite® 1200 (H), Amberlite® IRP-88, Amberlite® IRP-64, Dowex®1×2-100, 200, 400; 1×4-50, 100, 200, 400; 1×8-50, 100, 200, 400, and Duolite C-26.

### Glidants/Antisticking Agents:

Various useful glidants or antisticking agents include but are not limited to talc, silica derivatives, colloidal silicon dioxide and the like, and mixtures thereof.

### Lubricants:

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, Amberlite IRP88, castor oils, and waxes.

### Basifying Agents:

Suitable basifying agents that can be used for the purposes of this invention include, for example, sodium hydroxide, potassium hydroxide, calcium hydroxide, sodium carbonate, ammonia, diethanolamine, meglumine, lysine, arginine, ethanolamine, piperazine, trometamol and triethanolamine, ammonia, etc.

### Surfactants:

Various useful surfactants include but are not limited to sodium lauryl sulfate, dioctylsulfosuccinate, gelatin, casein, lecithin (phosphatides), dextran, gum acacia, cholesterol, tragacanth, hydroxypropyl methylcelluloses, hydroxypropylcelluloses, polyvinylpyrrolidones, polyoxyethylene alkyl ethers (e.g., macrogol ethers such as cetomacrogol 1000), polyoxyethylene castor oil derivatives, polyoxyethylene sorbitan fatty acid esters (such as the commercially available Tween™ products, e.g., Tween 20 and Tween 800 (ICI Speciality Chemicals)), polyethylene glycols (e.g., Carbowax 3550 and 934 (Union Carbide)), polyoxyethylene stearates, carboxymethylcellulose calcium, carboxymethyl cellulose sodium, methylcelluloses, hydroxyethylcelluloses, hydroxypropyl methylcellulose phthalates, magnesium aluminium silicate, triethanolamine, polyvinyl alcohols (PVA), poloxamers (e.g., Pluronic™ products F68 and F108Q, which are block copolymers of ethylene oxide and propylene oxide); poloxamines (e.g., Tetronic™ 908, also known as poloxamine 908, which is a tetrafunctional block copolymer derived from sequential addition of propylene oxide and ethylene oxide to ethylenediamine (BASF Wyandotte Corporation, Parsippany, N.J.)), Tetronic™ 15080 (T-1508) (BASF Wyandotte Corporation), PEG-derivatized phospholipids, PEG-derivatized cholesterols, PEG-derivatized cholesterol derivatives, PEG-derivatized vitamin A, PEG-derivatized vitamin E, lysozyme, random copolymers of vinylpyrrolidone and vinyl acetate, and the like.

### Colorants:

Various useful colorants include but are not limited to Food Yellow No. 5, Food Red No. 2, Food Blue No. 2, and the like, food lake colorants, and pigments such as iron oxides.

### Flavors:

Flavoring agents, which can be used in the present invention, include but are not limited to flavors having a natural or synthetic or semi synthetic origin like menthol, fruit flavors, citrus oils, peppermint oil, spearmint oil, and oil of wintergreen (methyl salicylate).

### Film-Forming Agents:

Various film forming agents that can be used include but are not limited to cellulose derivatives such as soluble alkyl- or hydroalkyl-cellulose derivatives such as methyl celluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc., acidic cellulose derivatives such as cellulose acetate phthalates, cellulose acetate trimellitates and methylhydroxypropylcellulose phthalates, polyvinyl acetate phthalates, etc.; insoluble cellulose derivatives such as ethyl celluloses and the like; dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates such as derivatives thereof (Eudragit™), chitosan and derivatives thereof, shellac and derivatives thereof, and waxes and fat substances.

If required, the films may contain additional adjuvants for coating processing such as plasticizers, polishing agents, colorants, pigments, antifoam agents, opacifiers, antisticking agents, and the like.

### Plasticizers:

Various plasticizers include but are not limited to castor oil, diacetylated monoglycerides, dibutyl sebacate, diethyl phthalate, glycerin, polyethylene glycols, propylene glycols, triacetin, and triethyl citrate. Also mixtures of plasticizers may be utilized. The type of plasticizer depends upon the type of coating agent. A plasticizer is frequently present in amounts ranging from about 5% (w/w) to 30 (w/w) based on the total weight of the film coating.

An opacifier like titanium dioxide may also be present in an amount ranging from about 10% (w/w) to about 20% (w/w) based on the total weight of the coating. When coloured tablets are desired then the colour is normally applied in the coating. Consequently, colouring agents and pigments may be present in the film coating. Various colouring agents include but are not limited to iron oxides, which can be red, yellow, black or blends thereof.

Antiadhesives can be used in the film coating process to avoid sticking effects during film formation and drying. An example of a useful antiadhesive for this purpose is talc. The antiadhesive is present in the film coating in an amount of about 5% (w/w) to 15% (w/w) based upon the total weight of the coating.

Suitable polishing agents include polyethylene glycols of various molecular weights or mixtures thereof, talc, surfactants (e.g. glycerol monostearate and poloxamers and poloxamer 188), fatty alcohols (e.g., stearyl alcohol, cetyl alcohol, lauryl alcohol and myristyl alcohol) and waxes (e.g., carnauba wax, candelilla wax and white wax). For example, polyethylene glycols having molecular weights of 3,000-20,000 can be employed.

In addition to above coating ingredients, sometimes pre-formulated coating products such as OPADRY™ (supplied by Colorcon), for example Opadry Blue 13B50579 can be used. These products require only mixing with a liquid before use.

### Solvents:

Various solvents used in the processes of preparing pharmaceutical formulations of the present invention include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran, and mixtures thereof.

In an embodiment the invention relates to processes for preparing pharmaceutical formulations comprising repaglinide or its derivatives. Processing is important especially when the active is a low dose potent active such as repaglinide, in order to achieve content uniformity. When processing is conducted in a fluid bed processor and includes a step of adding an active solution or dispersion over an excipient or a mixture of excipeints, top spray granulation is useful to achieve content uniformity.

In an embodiment the invention includes the use of top spray granulation in processes for preparing solid pharmaceutical formulations comprising repaglinide or its derivatives.

In another embodiment the invention includes the use of bottom spray granulation in processes for preparing solid pharmaceutical formulations comprising repaglinide or its derivatives.

Equipment suitable for processing the pharmaceutical formulations of the present invention include any one or more of mechanical sifters, blenders, roller compacters, compression machines, rotating bowls or coating pans, fluidized bed processors, etc.

### Processes for Preparing Pharmaceutical Formulations:

The present invention further relates to processes for manufacturing pharmaceutical formulations of the present invention, wherein an embodiment of a process comprises:

a) Sifting excipients through a sieve.

b) Dissolving or dispersing binder in a suitable solvent and optionally adding a surfactant or solubilizer.

c) Dispersing the active ingredient in the solution of b).

d) Optionally adding a solubilizer to the drug dispersion.

e) Optionally, adding a plasticizer to the drug dispersion.

f) Granulating step a) with drug-binder dispersion of e) using a fluid bed processor or rapid mixer granulator.

g) Drying the wet mass.

h) Sizing dried granules through a sieve. Sifting extragranular excipients through a sieve and adding to granules followed by blending.

i) Sifting lubricant through a sieve, adding to h) and blending.

j) Compressing lubricated blend from i) into a desired shape.

Pharmaceutical formulations can be tested for their physical properties such as weight variation, hardness, disintegration, friability, etc. Several devices have been used to test tablet hardness such as the Monsanto tester, the Strong-Cobb tester, the Pfizer tester, the Erweka tester, the Schleuniger tester, etc. Friability is determined by a Roche friabilator for 100 revolutions at 25 rpm. Disintegration testing for tablets can be performed in a USP tablet disintegration tester wherein a tablet is placed in a basket, which moves upward and downward in a 1 L beaker of a dissolution medium at 37° C.

Pharmaceutical formulations can be subjected to in vitro dissolution evaluations according to Test 711 “Dissolution” in United States Pharmacopoeia 29, United States Pharmacopeial Convention, Inc., Rockville, Md., 2005 (“USP”) to determine the rate at which the active substance is released from the dosage forms, and content of active substance can conveniently be determined in dissolution solutions using techniques such as high performance liquid chromatography. The pharmaceutical dosage forms of the present invention are intended for oral administration to a patient in need thereof.

In an embodiment the invention includes use of packaging materials such as containers and lids of high-density polyethylene (HDPE), low-density polyethylene (LDPE) and or polypropylene and/or glass, and blisters or strips composed of aluminium or high-density polypropylene, polyvinylchloride (PVC), polyvinylidene dichloride (PVDC), etc.

Further the invention also includes certain pharmacokinetic parameters resulting from administration of compositions of the present invention.

In an embodiment the invention includes pharmaceutical formulations comprising 2 mg of repaglinide and producing, following administration of a single dose to healthy humans, plasma Cₘₐₓ values of repaglinide about 29 ng/mL to about 50 ng/mL.

In an embodiment the invention includes pharmaceutical formulations comprising 2 mg of repaglinide and producing, following administration of a single dose to healthy humans, plasma AUC_{0-inf} values of repaglinide about 50 ng·hour/ml to about 80 ng·hour/ml.

In an embodiment the invention includes pharmaceutical formulations comprising 2 mg of repaglinide and producing, following administration of a single dose to healthy humans, plasma AUC₀₋ₜ values of repaglinide ranging from about 50 ng·hour/mL to about 80 ng·hour/mL.

In an embodiment, the invention includes pharmaceutical formulations comprising 2 mg of repaglinide and producing, following administration of a single dose to healthy humans, at least one of plasma repaglinide: Cₘₐₓ values less than about 50 ng/mL; AUC₀₋ₜ values less than about 80 ng·hour/mL; and AUC_{0-∞} values less than about 80 ng·hour/mL.

In an embodiment the invention relates to analytical methods for analysis of repaglinide-related impurities using high performance liquid chromatography (HPLC), wherein an embodiment of a method comprises:

Buffer solution: 4 g of potassium dihydrogen phosphate is dissolved in 1000 mL of water under sonication, pH is adjusted to 3.2 with orthophosphoric acid, and the solution filtered through a 0.45 μm hydrophilic membrane filter.

Mobile phase A: pH 3.2 buffer solution.

Mobile phase B: buffer solution and acetonitrile, mixed in the volume ratio of 3:7.

Diluent: 6.8 g of potassium dihydrogen phosphate is dissolved in 1000 mL of water under sonication, pH is adjusted to 1.8 with orthophosphoric acid, and the prepared pH 1.8 buffer solution is mixed with methanol in the volume ratio of 1:1.

Chromatographic System:

a) The liquid chromatograph is equipped with a 240 nm UV detector.

b) Column: 4.6 mm×250 mm, 5 μm, Zodiac C18 AQ.

c) Column temperature: 45° C.

d) Flow rate: 1 mL per minute.

e) Injection volume: 50 μL.

f) Run time: 90 minutes.

Mobile Phase Gradient Program:

| Time | Vol. % of Mobile | Vol. % of Mobile |
|---|---|---|
| (minutes) | Phase A | Phase B |
| 0.01 | 65 | 35 |
| 5 | 65 | 35 |
| 25 | 30 | 70 |
| 45 | 5 | 95 |
| 70 | 5 | 95 |
| 80 | 65 | 35 |
| 90 | 65 | 35 |

### Preparation of Test Sample:

30 tablets are crushed into a powder, and powder equivalent to 12.5 mg of repaglinide is transferred into a 25 mL volumetric flask. Then 15 mL of diluent is added and the mixture subjected to sonication between 20° C. and 25° C. for 30 minutes with intermittent shaking, and the volumetric flask is filled to volume with diluent. Finally a portion of the solution is subjected to centrifugation at 4000 RPM for 10 minutes, before injection.

Representative relative retention times (RRT, where repaglinide=1), limits of detection (LOD), and limits of quantification (LOQ) of various impurities are tabulated below.

| Impurity | RRT | LOD | LOQ |
|---|---|---|---|
| Impurity A | 0.16 | 0.001 | 0.003 |
| Impurity B | 0.43 | 0.001 | 0.004 |
| Impurity C | 0.54 | 0.007 | 0.024 |
| Impurity D | 1.35 | 0.004 | 0.014 |
| Desethyl impurity | 0.67 | 0.003 | 0.007 |
| Cyclized impurity I | 1.04 | ND | ND |
| Cyclized impurity II | 1.08 | ND | ND |
| Specified impurity I | 0.45 | ND | ND |
| Specified impurity II | 0.5 | ND | ND |
| Specified impurity III | 0.6 | ND | ND |

Certain specific aspects and embodiments of the invention will be further described in the following examples, which are provided only for purposes of illustration and are not intended to limit the scope of the invention in any manner.

### Example 1

### Properties of repaglinide used in the examples are shown in Table 1

TABLE 1

| Parameter | Value |
|---|---|
| Bulk density (g/ml) | 0.176 |
| Tapped density (g/ml) | 0.247 |
| Compressibility index | 29.95 |

Particle size distribution

| | |
|---|---|
| D₉₀ | <7 μm |
| D₅₀ | <4 μm |
| D₁₀ | 2 μm |

Densities were determined according to USP Test 616 “Bulk Density and Tapped Density,” and compressibility index is percentage change in volume induced by tapping a sample of fixed mass.

### Examples 2-3

### Repaglinide 2 mg Tablets

mg/Tablet

| Ingredient | Example 2 | Example 3 |
|---|---|---|
| Repaglinide | 2 | 2 |
| Meglumine | 1 | 1 |
| Poloxamer 188 | 0.6 | 0.6 |
| Glycerin | 1.2 | 1.2 |
| Povidone K 30 | 4 | 4 |
| Water* | 33 | 33 |
| Microcrystalline cellulose PH101 | 20 | 23.05 |
| Starch | 10 | 10 |
| Dicalcium phosphate anhydrous | 38 | 38 |
| Iron oxide red | 0.15 | 0.15 |
| Microcrystalline cellulose PH102 | 13.05 | 10 |
| Polacrilin potassium** | 4 | 4 |
| Magnesium stearate | 1 | 1 |

*Evaporates during processing.
**AMBERLITE ® IRP88 resin, sold by Rohm and Haas Co., Philadelphia, Pennsylvania U.S.A.

Manufacturing Process:

a) Povidone and poloxamer were dissolved in water with continuous stirring for about 15 minutes.

b) Repaglinide was dispersed in the solution of a), mixing uniformly for about 15 minutes.

c) Meglumine was added to the drug dispersion of b), and mixed uniformly for about 30 minutes.

d) Glycerin was added to the drug dispersion of c) and mixed uniformly for about 30 minutes.

e) Starch and iron oxide were sifted in geometrical proportions through an ASTM #80 mesh sieve.

f) Microcrystalline cellulose PH101 and dicalcium phosphate anhydrous and step e) were sifted through an ASTM #60 mesh sieve.

g) The step f) mixture was granulated using drug binder solution of step d) using top spray in a fluid bed processor and the granules were dried at an inlet air temperature at 60±5° C. for about 30 minutes, until loss on drying of granules at 105° C. was within the range of 0.5 to 3% w/w. The dried granules obtained were sifted through an ASTM #30 mesh sieve.

h) Microcrystalline cellulose PH102 and Amberlite IRP88 were sifted through an ASTM #40 mesh sieve and blended uniformly with step g) material in a double cone blender for 10 minutes.

i) Magnesium stearate was sifted through an ASTM #60 mesh sieve, added to the mixture of h) and blended for 5 minutes in a double cone blender.

j) The lubricated blend was compressed into tablets using 6 mm round, biconcave punches.

Physical parameters of the final blend with excipients and tablets prepared according to Example 2 are given in Table 2.

TABLE 2

| | |
|---|---|
| Parameter | Value |
| ASTM Sieve No. | Retained (%) |

Parameters for final blend

| | |
|---|---|
| 60 | 0.9 |
| 80 | 17 |
| 100 | 35.5 |
| <100 | 46.6 |

Physical parameters for tablets

| | |
|---|---|
| Hardness (Kilopond, “kp”) | 4-5.5 |
| Disintegration time (minutes) | 1.3 |
| Content uniformity | 92.6 |
| % Relative standard deviation | 1.5 |

Dissolution profiles of the Example 2 product and PRANDIN® 2 mg repaglinide tablets were compared and the data are shown in Table 3. Dissolution conditions: 900 mL of pH 5.0 citrate buffer, USP Type II apparatus, 75 rpm stirring.

TABLE 3

| | Cumulative % of Drug Released | | |
|---|---|---|---|
| Time (minutes) | PRANDIN ® | Example 2 | Example 3 |
| 10 | 95 | 76 | 97 |
| 20 | 95 | 80 | 100 |
| 30 | 95 | 82 | 101 |
| 45 | 94 | 83 | 101 |

Content uniformity for ten tablets prepared according to Example 3 was studied at different stages of a compression run, using the method of USP Test 905 “Uniformity of Dosage Units.” The stages were the beginning, the middle, and the end of the compression run. Results in terms of average drug content (as a percentage of the label drug content) and % RSD (relative standard deviation) of the drug content are shown in Table 4.

TABLE 4

| Parameter | Beginning | Middle | End |
|---|---|---|---|
| Average Drug Content (%) | 100 | 100 | 100 |
| RSD (%) | 1.4 | 1.7 | 1.5 |

### Example 4

### Stability Studies

Tablets prepared according to Example 2 were packaged in three different packages and stored under accelerated stability conditions (40° C. and 75% relative humidity) for two months, and samples were analyzed at the time of packaging and at intervals for related impurities (percent of label drug content), dissolution, and purity (percent of label drug content). The results tabulated in Table 5

### Example 2A

### Tablets Packed in Closed HDPE Containers with Desiccant (Silica gel)

### Example 2B

### Tablets Packaged in Aluminum Foil Blisters

### Example 2C

### Tablets Packaged in PVC-PVDC Blisters

TABLE 5

| | Example 2A | | | Example 2B | | Example 2C | |
|---|---|---|---|---|---|---|---|
| Parameter | Initial | 1 Mo. | 2 Mos. | 1 Mo. | 2 Mos. | 1 Mo. | 2 Mos. |
| Purity | 98 | 95.7 | 97.6 | 95.6 | 96.5 | 95.7 | 98 |
| Cyclic Impurity 2 | ND* | 0.05 | 0.09 | 0.04 | 0.01 | 0.04 | 0.008 |
| Unidentified Impurity | 0.03 | 0.04 | 0.04 | 0.04 | 0.04 | 0.05 | 0.04 |
| Total Impurities | 0.13 | 0.24 | 0.21 | 0.23 | 0.25 | 0.27 | 0.27 |
| Dissolution (%) | 86.8 | 88 | 76 | 83 | 81 | 84 | 81 |

*ND: not detected.

Dissolution results are percentages of contained repaglinide dissolved in 900 ml of 0.1 N HCl within 30 minutes.

Prandin® tablets and tablets prepared according to Example 3 were packaged in aluminum foil blisters and stored under accelerated stability conditions (40° C. and 75% relative humidity) for six months. Samples were analyzed at the time of packaging and at storage intervals for repaglinide-related impurities (percent of label drug content), dissolution (dissolution conditions as in Example 2), assay (percent of label drug content), water by KF, disintegration in water and friability. The results are tabulated in Table 6.

TABLE 6

| | PRANDIN ® | | | | Example 3 | | | |
|---|---|---|---|---|---|---|---|---|
| Parameter | Initial | 1 Mo. | 3 Mos. | 6 Mos. | Initial | 1 Mo. | 3 Mos. | 6 Mos. |
| Drug assay | 97.5 | NA* | 98.5 | 102.4 | 100.4 | 102.9 | 102.9 | 101.8 |
| Impurity A | ND** | NA | ND | ND | ND | 0.001 | 0.002 | 0.002 |
| Impurity B | ND | NA | ND | ND | ND | ND | ND | ND |
| Impurity C | ND | NA | ND | ND | 0.009 | ND | 0.03 | 0.02 |
| Impurity D | ND | NA | ND | ND | ND | ND | 0.009 | 0.01 |
| Desethyl impurity | ND | NA | ND | ND | 0.004 | 0.009 | 0.003 | 0.05 |
| Specified Impurity I | ND | NA | ND | ND | 0.02 | 0.02 | 0.03 | 0.04 |
| Specified Impurity II | ND | NA | ND | ND | 0.04 | 0.03 | 0.02 | 0.05 |
| Specified Impurity | ND | NA | ND | ND | 0.06 | 0.08 | 0.11 | 0.16 |
| III | | | | | | | | |
| Cyclized impurity I | ND | NA | ND | ND | 0.04 | 0.08 | 0.03 | 0.02 |
| Cyclized impurity II | ND | NA | ND | ND | ND | 0.004 | 0.1 | 0.13 |
| Highest unidentified | 0.0505 | NA | 0.0351 | 0.0345 | 0.05 | 0.04 | 0.02 | 0.06 |
| impurity | | | | | | | | |
| Total impurities | 0.1345 | NA | 0.1127 | 0.071 | 0.35 | 0.42 | 0.52 | 0.75 |
| Dissolution (%) | 96 | NA | — | — | 97 | 98 | 96 | 98 |
| Water by KF (%) | 3.4 | NA | — | — | 2.9 | 3.4 | 4.6 | 3.4 |
| Disintegration time | <2 | NA | — | — | <5 | <4 | <3 | 5 |
| (minutes) | | | | | | | | |
| Friability (%) | — | NA | — | — | 0.02 | 0 | 0 | 0 |

*NA: Not performed.
**ND: Not detected.

### Example 5

### Pharmacokinetic Parameters of Formulations Prepared According to Example 2

Tablets were evaluated in a open label, balanced, randomized, two treatment, two-period, two-sequence, single-dose, crossover bioequivalence study in healthy, adult male human subjects under fasting conditions, involving administration of the test product and the commercial reference product PRANDIN 2 mg repaglinide tablets to 14 fasting healthy human volunteers, and plasma concentrations were determined at intervals after dosing.

The following parameters were calculated:
AUC₀₋ₜ=the area under plasma concentration versus time curve, from time zero to the time of the last measurable concentration.

AUC_{0-∞} m=area under the plasma concentration versus time curve, from time zero to infinity.

Cₘₐₓ=maximum plasma concentration.

The results of these pharmacokinetic parameters in the fasting study were calculated and are summarized in Table 7.

TABLE 7

| | Example 2 | PRANDIN | (T ÷ R) × | 90% |
|---|---|---|---|---|
| Parameter | (T) | (R) | 100 | C.I. (%) |
| AUC₀₋ₜ (ng · hour/mL) | 63.018 | 59.17 | 106.5 | 101.88 |
| AUC_{0-∞} (ng · hour/mL) | 64.197 | 60.707 | 105.75 | 100.81 |
| Cₘₐₓ (ng/mL) | 40.283 | 37.325 | 107.93 | 87.10 |

### Example 6

### Repaglinide 1 mg Tablets

| Ingredient | mg/Tablet |
|---|---|
| Repaglinide | 1 |
| Meglumine | 0.5 |
| Poloxamer 188 | 0.3 |
| Glycerin | 0.6 |
| Povidone | 4 |
| Water* | 50 |
| Microcrystalline cellulose PH101 | 25.45 |
| Starch | 10 |
| Calcium hydrogen phosphate anhydrous | 38 |
| Iron oxide yellow | 0.15 |
| Microcrystalline cellulose PH102 | 10 |
| Amberlite IRP88 | 4 |
| Magnesium stearate | 1 |

*Evaporates during processing.

Manufacturing process is similar to that of Example 2.

Tablets prepared according to Example 6 and PRANDIN® tablets were tested for dissolution characteristics and the data are tabulated in Table 8. Dissolution conditions were the same as in Example 2.

TABLE 8

| Time (minutes) | PRANDIN ® 1 mg | Example 6 |
|---|---|---|
| 10 | 81 | 91 |
| 20 | 90 | 99 |
| 30 | 90 | 101 |
| 45 | 94 | 102 |

### Example 7-8

### Repaglinide 0.5 mg Tablets

mg/Tablet

| | | |
|---|---|---|
| Ingredient | Example 7 | Example 8 |

| | | |
|---|---|---|
| Repaglinide | 0.5 | 0.5 |
| Meglumine | 0.25 | 0.25 |
| Poloxamer 188 | 0.15 | 0.15 |
| Glycerin | 0.3 | 0.3 |
| Povidone K30 | 4 | 4 |
| Water* | 15 | 15 |
| Microcrystalline cellulose PH101 | 25 | 26.8 |
| Starch | 10 | 10 |
| Calcium hydrogen phosphate anhydrous | 38 | 38 |
| Microcrystalline cellulose PH102 | 11.8 | 10 |
| Amberlite IRP88 | 4 | 4 |
| Magnesium stearate | 1 | 1 |

*Evaporates during processing.

Manufacturing process is similar to that of Example 2, except that there is no colorant and starch from step e) is incorporated into step f).

### Example 9

### Repaglinide 2 mg Tablets

| Ingredient | mg/Tablet |
|---|---|
| Repaglinide | 2 |
| Meglumine | 1 |
| Poloxamer 188 | 0.572 |
| Glycerin | 1.4 |
| Povidone K 30 | 1.972 |
| Water* | 30 |
| Starch B700 | 30 |
| Dibasic calcium phosphate anhydrous | 20 |
| Microcrystalline cellulose (Avicel PH101) | 36.93 |
| Iron oxide red | 0.125 |
| Amberlite IRP88 | 5 |
| Magnesium stearate | 1 |

*Evaporates during processing.

Manufacturing Process:

a) Povidone K 30 was added to water and stirred to dissolve completely.

b) Repaglinide was added to step a) and dispersed to form a homogenized suspension.

c) Meglumine and poloxamer were added to step b) and stirred for 10 minutes.

d) Glycerin was added to drug mixture of step c) and stirred for 15 minutes.

e) Starch and iron oxide red were sifted together in geometric proportions through an ASTM #80 mesh sieve.

f) Blend of e), microcrystalline cellulose (Avicel PH101), and dibasic calcium phosphate anhydrous were sifted through an ASTM #60 mesh sieve.

g) Blend of f) was granulated with step d) in a rapid mixer granulator (Model FR042, supplied by Sams Machines Pvt. Ltd., Mumbai, India) at a slow speed and dried to obtain a loss on drying at 50° C. less than 3%.

h) Above dried granules were sifted through an ASTM #30 mesh sieve.

i) Polacrilin potassium was sifted through an ASTM #40 mesh sieve, added to step g) and blended uniformly in a double cone blender for 10 minutes.

j) Magnesium stearate was sifted through an ASTM #60 mesh sieve, added to step i) and blended uniformly for 5 minutes in a double cone blender.

k) Lubricated blend was compressed into tablets using 6 mm round punches.

Comparative dissolution profiles of Example 7 and PRANDIN® 2 mg repaglinide tablets were obtained and data are in Table 9. Dissolution conditions: 900 ml of pH 5.0 citrate buffer as medium, USP Type II apparatus, 75 rpm stirring.

TABLE 9

Cumulative % of Drug Released

| Time (minutes) | PRANDIN ® | Example 9 |
|---|---|---|
| 10 | 80 | 92 |
| 20 | 90 | 99 |
| 30 | 92 | 99 |

We claim:

1. A process for preparing a pharmaceutical formulation, comprising granulating at least one pharmaceutically acceptable excipient with a solution or dispersion comprising repaglinide, or a derivative thereof, and optionally a basifying agent.

2. The process of claim 1, wherein a basifying agent comprises meglumine.

3. The process of claim 1, wherein a solution or dispersion further comprises at least one of a binder and a surfactant.

4. The process of claim 3, wherein a binder comprises a polyvinylpyrrolidone.

5. The process of claim 3, wherein a surfactant comprises a poloxamer.

6. The process of claim 1, wherein granulation is conducted in a fluid bed granulator.

7. The process of claim 1, wherein granulation is conducted in a rapid mixer granulator.

8. The pharmaceutical formulation prepared by the process of claim 1, wherein relative standard deviation of repaglinide content in the formulation is not greater than about 6.

9. The pharmaceutical formulation prepared by the process of claim 1, containing total repaglinide-derived impurities at less than about 2 percent of the label content of repaglinide.

10. The pharmaceutical formulation prepared by the process of claim 1, containing 2 mg of repaglinide and producing, following administration of a single dose to healthy humans, at least one of plasma repaglinide: Cₘₐₓ values less than about 50 ng/mL; AUC₀₋ₜ values less than about 80 ng·hour/mL; and AUC_{0-∞} values less than about 80 ng·hour/mL.

11. A process for preparing a pharmaceutical formulation, comprising granulating at least one pharmaceutically acceptable excipient with a solution or dispersion comprising repaglinide, or a derivative thereof, and a basifying agent comprising meglumine, wherein granulation is conducted in a fluid bed granulator.

12. The process of claim 11, wherein a solution or dispersion further comprises at least one of a binder and a surfactant.

13. The process of claim 12, wherein a binder comprises a polyvinylpyrrolidone.

14. The process of claim 12, wherein a surfactant comprises a poloxamer.

15. The process of claim 11, wherein a solution or dispersion further comprises a binder and a surfactant.

16. A process for preparing a pharmaceutical formulation, comprising granulating at least one pharmaceutically acceptable excipient with a solution or dispersion comprising repaglinide, or a derivative thereof, and a basifying agent comprising meglumine, wherein granulation is conducted in a rapid mixer granulator.

17. The process of claim 16, wherein a solution or dispersion further comprises at least one of a binder and a surfactant.

18. The process of claim 17, wherein a binder comprises a polyvinylpyrrolidone.

19. The process of claim 17, wherein a surfactant comprises a poloxamer.

20. The process of claim 16, wherein a solution or dispersion further comprises a binder and a surfactant.
